(19) United States
(12) Patent Application Publication
(10) Pub. No.: US 2020/0323937 A1
(43) Pub. Date: Oct. 15, 2020

(54) ORALLY DISINTEGRATING TABLET OF CANNABIS EXTRACT AND METHOD OF MAKING

(71) Applicant: Hemp Bioscience LLP, Colorado Springs, CO (US)

(72) Inventor: Stephen D. Ochs, Colorado Springs, CO (US)

(21) Appl. No.: 16/847,142

(22) Filed: Apr. 13, 2020

Related U.S. Application Data
(60) Provisional application No. 62/832,326, filed on Apr. 11, 2019.
(60) Provisional application No. 62/860,349, filed on Jun. 12, 2019.

(51) Int. Cl. A61K 36/185; A61K 9/16; A61K 9/00

(52) U.S. Cl. CPC A61K 36/185; A61K 9/0053; A61K 9/1652

(57) ABSTRACT

An orally disintegrating tablet comprising Cannabis sativa L. extracts which are substantially free of tetrahydrocannabinol (THC), wherein the extracts have an average diameter of less than 1 micrometer.

### BACKGROUND OF THE INVENTION

### 1. Field of the Invention

The invention generally relates to an orally disintegrating tablet that includes whole plant hemp extracts and methods of making the orally disintegrating tablet.

### 2. Description of the Relevant Art

The plant Cannabis sativa L. (the “cannabis plant”) is best known for its psychoactive substituents and its medicinal effects. Pharmacologically, the principal psychoactive constituent of cannabis plants is tetrahydrocannabinol (delta9-THC). Cannabidiol (CBD) is another cannabinoid present in the cannabis plant and is known to have many health benefits such as antidepressant, anti-anxiety, anti-inflammatory, and neuroprotective effects. In contrast to THC, CBD and the other non-THC cannabinoids of hemp do not exert psychoactive effects.

Oral dosage forms of cannabis plant extracts typically suffer from low bioavailability. After an oral dosage of cannabis extract is swallowed, it is absorbed by the digestive system and enters the liver. The liver metabolizes the active components in the cannabis extract (e.g., CBD) to such an extent that only a small amount of active drug emerges from the liver to the rest of the circulatory system. This “first pass metabolism” contributes to the low bioavailability of the active components of the cannabis extract. Alternative routes of administration, such as sublingual or orally disintegrating tablets avoid the first-pass metabolism because the active components are absorbed directly into systemic circulation through the oral mucosa—referred to as being pre-gastric absorption.

It would be advantageous to develop an efficient and effective cannabis-based product that overcomes both the gastric acid breakdown of the stomach as well as the first pass hepatic metabolism drawbacks associated with traditional oral administration of medicines.

### SUMMARY OF THE INVENTION

In an embodiment, an orally disintegrating tablet includes Cannabis sativa L. extracts which are substantially free of tetrahydrocannabinol (THC). The Cannabis sativa L. extracts, have an average particle size of between about 25 nanometers to about 50 nanometers. The Cannabis sativa extracts may be uniformly distributed throughout the tablet.

The tablet may further include microcrystalline cellulose, a carbohydrate, and a disintegrant. The microcrystalline cellulose may be silicified microcrystalline cellulose. The carbohydrate may be xylitol. The disintegrant may be a polyvinyl pyrrolidone (PVP) polymer. The tablet may also include a lubricant and/or a flavoring agent.

The tablet may have a friability of not more than 1% according to USP 1216. The tablet may have a breaking force sufficient to allow the tablet to be pushed through foil blister packaging without breaking according to USP 1217. The tablet may have a disintegration of not more than 1 minute in water according to USP 701.

In an embodiment, an orally disintegrating tablet includes Cannabis sativa extracts which are substantially free of tetrahydrocannabinol (THC); microcrystalline cellulose; xylitol; and soy free crospovidone.

In an embodiment, a method of preparing an orally disintegrating tablet includes: obtaining Cannabis sativa extracts which are substantially free of tetrahydrocannabinol (THC); milling the Cannabis sativa extracts to an average particle size of between about 25 nanometers to about 50 nanometers; blending the milled Cannabis sativa extracts with microcrystalline cellulose, a carbohydrate, and a disintegrant to form a tablet composition; and compressing the tablet composition to form the orally disintegrating tablet.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Any specific examples that are included herein are intended to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In one embodiment, an orally disintegrating tablet includes Cannabis sativa L. (“cannabis plant”) extracts which are substantially free of tetrahydrocannabinol (delta9-THC). The cannabis plant extracts have an average particle size of less than 1 micron after the proprietary extraction process on the biosource raw material. The extracts may have an average particle size of less than 500 nanometers. The extracts may have an average particle size of less than 100 nanometers. The extracts may have an average particle size of between about 25 nanometers to about 50 nanometers.

Cannabis plant extracts may be obtained using a variety of different methods. In one embodiment, cannabis plant extracts are obtained using a super critical gas/liquid carbon dioxide extraction process. The extraction process removes substantially all of the tetrahydrocannbinol (THC) psychoactive cannabinoids so that an extract is obtained that is substantially free of THC. The resulting extract includes CBD, as well as other phytocannabinoids and cannabinoids, along with dozens of terpenes and flavonoids. The extract is then milled to nano sized particles having an average particle size of less than 1 micron. The resulting powder is used as the active component of the tablet.

The tablet is formed by blending the milled Cannabis sativa extracts with microcrystalline cellulose, a carbohydrate, and a disintegrant to form a tablet composition. Microcrystalline cellulose, as used herein, refers to cellulose, typically obtained from wood pulp, that has been milled to sub-millimeter particle size. In one embodiment, microcrystalline cellulose used in forming the table composition has a particle size of between 5 μm to 900 In preferred embodiments, the microcrystalline cellulose has a particle size between 100 μm and 200 μm.

In some embodiment, silicified microcrystalline cellulose (“SMCC”) may be used to form the tablet. Silicified microcrystalline cellulose is composed of a mixture of colloidal silicon dioxide with microcrystalline cellulose. The microcrystalline cellulose to colloidal silicon dioxide ratio may be between 80:20 to 98:2. An exemplary SMCC material may be SMCC HD90 sold as PROSOLV SMCC by JRS Pharma.

The tablet may further comprise a carbohydrate material. The carbohydrate material may serve as both a sweetener and a binder. Exemplary carbohydrate materials include, but are not limited to, dextrose, starch, sucrose, xylitol, lactitol, and sorbitol.

To promote the oral disintegration of the tablet, a disintegrant may be added to the tablet composition. In a preferred embodiment, the disintegrant is a polyvinyl pyrrolidone (PVP) polymer. The term “polyvinylpyrrolidone” or “PVP” refers to a polymer, either a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. The cross-linked homopolymer is insoluble and is generally known in the pharmaceutical industry under the designations polyvinylpolypyrrolidone, crospovidone and PVP. The copolymer vinylpyrrolidone-vinyl acetate is generally known in the pharmaceutical industry under the designations Copolyvidon(e), Copolyvidonum or VP-VAc.

In addition to the primary tablet composition components, one or more functional excipients such as lubricants, fillers, antioxidants, buffering agents, alkalinizing agents, acidifying agents, disintegrants, diluents, sweeteners, chelating agents, colorants, flavorants, surfactants, solubilizers, wetting agents, stabilizers, enhancers, bioadhering/mucus retaining agents, preservatives, absorbents, cross-linking agents, bioadhesive polymers, retardants, pore formers, osmotic agents crystallization inhibitors, poloxamers, and fragrance may be present. Such compounds are, generally, functionally inert and are therefore optional components of the tablet.

Lubricants may be particularly useful for forming tablets. Lubricants are compounds that prevent, reduce or inhibit adhesion or friction of materials. Exemplary lubricants include, but are not limited to magnesium stearate, sodium stearate, stearic acid (stearin), hydrogenated oil, waxes, colloidal silicon dioxide, micronized polyoxyethylene glycol, sodium stearyl fumarate and combinations thereof.

Flavorants are also particularly useful for helping to mask the taste of the other tablet components.

Once the tablet composition has been formed, the composition may be, optionally, blended and milled to nanosized particles. The tablet composition may be introduced into a tablet press and compressed into a tablet form. The resulting tablet will have cannabis extracts distributed throughout the tablet. The pressure of the tablet compression process and the ratio of the various tablet components may be adjusted to create a tablet having: a friability of not more than 1% according to USP 1216; a breaking force sufficient to allow the tablet to be pushed through foil blister packaging without breaking according to USP 1217, and a disintegration of not more than 1 minute in water according to USP 701.

In a specific embodiment, an orally dissolving tablet containing hemp extracts is composed as follows:
12.3%—Hemp Extract
24.5%—Silicified microcrystalline cellulose
46.5%—Xylitol
13.5%—Soy free Crospovidone
3%—Flavorants
0.2%—Lubricant

The resulting tablets, formed according to the methods and compositions described herein, offer a number of difference and advantages over prior art tablets.

The tablet composition is milled and blended to nanometer sized particles in order to (1) obtain a reliably homogenous mixture of both the cannabis extracts and the excipients having comparable particle size for successful formulation and (2) to make the resulting mixture more hydrophilic to overcome the natural lipophilic nature of the cannabis extracts, which leads to a superior systemic absorption rate (e.g. as to percentage of administered API dose).

The cannabis extracts are uniformly present throughout the formulation and then dry pressed into a tablet using variable KN compression forces dependent on relative ambient humidity and temperature.

The active ingredients are a whole plant hemp extract less any tetrahydrocannabinoids (mostly THC) removed by supercritical gas/liquid CO₂ extraction, as opposed to a pure CBD isolate or an undefined, random aggregate of phytocannabinoids. Because of this, the formulation contains the whole hemp plant natural botanical profile including all naturally present cannabinoids (except THC and its psychoactive analogs), all terpenes and all flavonoids in addition to naturally occurring steroidal molecules all normally found in the hemp botanical.

Our milled and homogenously blended tablet composition, that is composed of nanometer particle sizing (e.g., having an average particle size of less than 1 micron) physically allows an improved tissue absorption amount as measured by plasma bioavailability (i.e. gastric/hepatic first pass of 10-25% verses up to 45-50% for orally dissolving tablet absorption of initial dose amount) in the oral (pharynx and tongue) soft tissue planes. It is believed that the size parity of the active components and the epithelial cells (i.e. the average epithelial cell measures 7-21 nanometers) lead to a reduction of the rate limiting cellular physical gradient. This may be used to allow for self-titration to the patient's desired subjective end-point in both clinical effect and symptom improvement.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Orally disintegrating tablets were made by compressing a table composition into a tablet. The tablet composition comprises Cannabis sativa L. extracts, microcrystalline cellulose (Prosolv HD90), a carbohydrate (Xylitol), a disintegrant (Crospovidone), flavorants (Mint and Lemon) and a lubricant (Magnesium Stearate). The amounts of each component present in the tablet composition is presented in Table 1.

### Table Composition—Sample 1

For formation of the tablet composition a blender (1 cubic foot) and a mill (Uni-Mill M05 with a round stator and R032 Screen) were used. Prosolv HD90 and Hemp Extract were passed through a #20 screen into a poly-lined container. The Prosolv/Hemp mixture was blended in the mill with an R032 screen at 1000±20 rpm into a poly-lined container. The blended Prosolv/Hemp mixture was added to the blender and to the mixture Crospovidone, Lemon, Mint and Xylitol (in that order) were added. The mixture was blended for 288 rotations. The blender was emptied through a #20 screen adding the Magnesium Stearate to the top of the screen approximately halfway through the process of emptying the blender. The contents of the blender were emptied, through the screen, into a poly-lined container. The resulting mixture of all components was added back to the blender and blended for 144 rotations. The reblended material was discharged into a poly-lined container and used as the table composition for tablet formation.

TABLE 1

| | Tablet | Weight | % |
|---|---|---|---|
| Ingredient | (mg) | (g) | Component |
| 20% Hemp Extract | 50 | 1197.5 | 12.25% |
| Xylitol | 190 | 4550.5 | 46.57% |
| Prosolv HD90 | 100 | 2395.0 | 24.50% |
| Crospovidone | 55 | 1317.2 | 13.48% |
| Mint | 8 | 191.6 | 1.96% |
| Lemon | 4 | 95.8 | 0.98% |
| Mg Stearate | 1 | 24.0 | 0.25% |
| TOTAL | 408 | 9771.6 | |

### Compression

Tablets were formed using a rotary tablet press. The tablet composition formed in the previous step, was added to the rotary press hopper. The tablet composition was equilibrated to the target parameters and pressed into tablets. The resulting tablets were tested for weight, thickness, and hardness. The results are presented in Table 2 as Experiment (Exp.) #1. The tablets produced had a hardness which is sufficient for oral use, but were too soft for use in a blister pack.

### Example 2

### Study to Improve Hardness of Tablets

A study was conducted to determine modifications to the tablet composition that would result in improved hardness of the tablets. The process and formulation development were conducted using approximately 50 grams of the active component (Sample 1). Tablet specifications were set using the Carver manual press where compression forces and dwell time vary to that of the rotary press. Rotary tablet presses have much shorter dwell times (amount of time the tools are under pressure).

Increasing the percentage of PROSOLV HD90 in the formulation increased the tablet hardness. These trials were hand pressed on the rotary press and showed that the tablet hardness could be increased by adding additional PROSOLV HD90 to the current formulation (Exp. 2 and 3). Additional Crospovidone was also added to promote tablet disintegration (Exp. 4). Results from the trials are shown below in Table 2.

TABLE 2

| | | Weight | Thickness | Hardness |
|---|---|---|---|---|
| Exp. # | Formulation | (mg) | (mm) | (K_{P}) |
| 1 | Sample 1 | 393.0 | 3.59 | 1.1 |
| 2 | Sample 1 + 40 mg | 422.1 | 3.68 | 2.6 |
| | Prosolv | | | |
| 3 | Sample 1 + 80 mg | 463.8 | 3.84 | 4.8 |
| | Prosolv | | | |
| 4 | Sample 1 + 70 mg | 462.2 | 3.82 | 5.1 |
| | Prosolv + 10 mg | | | |
| | Crospovidone | | | |

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An orally disintegrating tablet comprising Cannabis sativa L. extracts which are substantially free of tetrahydrocannabinol (THC), wherein the extracts have an average particle size of less than 1 micron.

2. The orally disintegrating table of claim 1, wherein the extracts have an average particle size of less than 500 nanometers.

3. The orally disintegrating table of claim 1, wherein the extracts have an average particle size of less than 100 nanometers.

4. The orally disintegrating table of claim 1, wherein the extracts have an average particle size of between about 25 nanometers to about 50 nanometers.

5. The tablet of claim 1, further comprising microcrystalline cellulose, a carbohydrate, and a disintegrant.

6. The tablet of claim 5, wherein the microcrystalline cellulose is silicified microcrystalline cellulose.

7. The tablet of claim 5, wherein the carbohydrate is xylitol.

8. The tablet of claim 5, wherein the disintegrant is a polyvinyl pyrrolidone (PVP) polymer.

9. The tablet of claim 1, wherein the Cannabis sativa extracts are uniformly distributed throughout the tablet.

10. The tablet of claim 1, wherein the tablet has a friability of not more than 1% according to USP 1216.

11. The tablet of claim 1, wherein the tablet has a breaking force sufficient to allow the tablet to be pushed through foil blister packaging without breaking according to USP 1217.

12. The tablet of claim 1, wherein the tablet has a disintegration of not more than 1 minute in water according to USP 701.

13. A method of preparing an orally disintegrating tablet, comprising:
obtaining Cannabis sativa extracts which are substantially free of tetrahydrocannabinol (THC);
milling the Cannabis sativa extracts to an average particle size of less than 1 micron;
blending the milled Cannabis sativa extracts with microcrystalline cellulose, a carbohydrate, and a disintegrant to form a tablet composition; and
compressing the tablet composition to form the orally disintegrating tablet.

14. The method of claim 13, wherein the microcrystalline cellulose is silicified microcrystalline cellulose.

15. The method of claim 13, wherein the carbohydrate is xylitol.

16. The method of claim 13, wherein the disintegrant is a polyvinyl pyrrolidone (PVP) polymer.

17. The method of claim 13, wherein the type and amount of the microcrystalline cellulose, the carbohydrate, and the disintegrant, are selected to produce a tablet that has a friability of not more than 1% according to USP 1216.

18. The method of claim 13, wherein the type and amount of the microcrystalline cellulose, the carbohydrate, and the disintegrant, are selected to produce a tablet that has a breaking force sufficient to allow the tablet to be pushed through foil blister packaging without breaking according to USP 1217.

19. The method of claim 13, wherein the type and amount of the microcrystalline cellulose, the carbohydrate, and the disintegrant, are selected to produce a tablet that has a disintegration of not more than 1 minute in water according to USP 701.
